(19) United States
(12) Patent Application Publication
(10) Pub. No.: US 2004/0175290 A1
(43) Pub. Date: Sep. 9, 2004

(54) Treatment of air and surfaces in a food processing plant

(75) Inventors: Robert Scheir, Sherman Oaks, CA (US); Robert Culbert, Manhattan Beach, CA (US)

Correspondence Address: SOCAL IP LAW GROUP, 310 N. WESTLAKE BLVD. STE 120, WESTLAKE VILLAGE, CA, 91362, US

(73) Assignee: Steril-Aire USA, Inc., Cerritos, CA (US)

(21) Appl. No.: 10/383,199

(22) Filed: Mar. 6, 2003

(51) Int. Cl. A01N 1/00; C23F 11/00

(52) U.S. Cl. 422/24; 422/22; 422/1

(57) ABSTRACT

There are disclosed systems and methods to treat surfaces of food processing machinery and air in a food processing plant. A method of treating a food processing station may comprise positioning a light emitting treatment system so the output of a germicidal lamp included in the light emitting treatment system will fall on a desired surface of the food processing station. The germicidal lamp of the light emitting treatment system may be energized to emit substantially uniformly distributed ultraviolet radiation across a surface of the food processing station.

### NOTICE OF COPYRIGHTS AND TRADE DRESS

A portion of the disclosure of this patent document contains material which is subject to copyright protection. This patent document may show and/or describe matter which is or may become trade dress of the owner. The copyright and trade dress owner has no objection to the facsimile reproduction by any one of the patent disclosure as it appears in the Patent and Trademark Office patent files or records, but otherwise reserves all copyright and trade dress rights whatsoever.

### BACKGROUND OF THE INVENTION

1. Field of the Invention

The present invention relates to treatment of air and surfaces in a food processing plant with ultraviolet radiation.

2. Description of Related Art

One mature industry that is economically sensitive to costs is the food processing industry. Because of its competitive nature, food processing systems must be inexpensive to install. Of a more global interest though, is the cost to operate and maintain food processing systems. Often, a plant owner will replace aging components or an entire system as the reduction in operating and maintenance costs can offset the retrofit cost, sometimes in a matter of months.

Broad social and health policies also favor healthier food processing systems. In these days of heightened food safety awareness, it has become even more important to prevent illnesses from processed foods.

Food processing plants are typically comprised of a number of stations. Food products and packaging may be moved manually between stations, or there may be stations such as conveyors which move the food products and packaging to other stations. In food processing plants, considerable effort is made to minimize contamination of the food products, packaging, and the equipment in the plant.

Several methods of controlling contamination in a food processing plant include chemical treatments, temperature, and various forms of irradiation. These methods typically provide fixed-position apparatus which can treat only a single station, or less. Because these treatments can be harmful to people and equipment, the exposure of these treatments must be carefully controlled. As a result, most treatment apparatus are custom-designed for specific stations and forms of treatment.

Organic matter often impinges and collects on various parts of the food processing stations. Though the surfaces of the food processing equipment may appear to be smooth, in fact, when viewed under a microscope, they can be seen to have an irregular and somewhat pitted surface. The organic matter can therefore adhere easily to the surfaces.

Environmental conditions in food processing plants can also favor growth of microorganisms. Many areas of a food processing plant may be dark and warm. Though some stations may be quite cold during operation, they may have varying cycles of cooling and warming. Water and food product particulates often appear throughout a plant, providing excellent sources of nutrients and safe harbors for microorganisms.

Food processing plants also have facilities for removing and moving food products and water, including drains and drain pans. Drains and drain pans become a growth environment for mold and bacteria. Water, excess food, food processing byproducts, and other waste flow into the drains and drain pans. They may carry organic matter, mold spores and bacteria. The drain pans are by design points of collection for water, and the standing water and most areas in a drain pan are excellent environments for microbial growth. Organic matter and microbial activity progressively clog the drain pan's drain, exacerbating the problems and seriously impeding the primary functions of the drain pan and drain. The drain pan may also act as a secondary source of contamination of the food processing stations.

Altogether, these consequences produce an environment in which microorganisms, including molds, bacteria and viruses, can grow and thrive. Over time, a food processing station can become encrusted with microorganism activity. The microorganisms, their spores and products of metabolism are easily entrained into the air and onto food products and packaging.

Germicidal lamps emit ultraviolet light at the primary and secondary emission lines of mercury (254 nm and 185 nm). At mercury's 185 nm line, ozone is created. Ozone has strict threshold limit values due to its strong oxidative properties and potential harm to humans. Despite the clear benefits of germicidal lamps, problems such as ozone, decreased output in low temperatures and moving air, and the resulting short life have prevented their use in all but the most friendly of environments.

For further information concerning improvements in germicidal lamps which are directed to overcoming such problems, reference is made to U.S. Pat. No. 5,334,347 entitled “Electric Discharge Device,” U.S. Pat. No. 5,866,076 entitled “Single-Ended Germicidal Lamp for HVAC Systems,” and U.S. Pat. No. 6,280,686 entitled “Control of Health Hazards in an Air Handler,” which are co-owned with this application.

### DESCRIPTION OF THE DRAWINGS

The present invention will be described by way of exemplary embodiments, but not limitations, illustrated in the accompanying drawings in which like references denote similar elements.

FIG. 1A is a side elevation of a light emitting treatment system in accordance with the invention.

FIG. 1B is a view of an embodiment of a reflector housing of a light emitting treatment system in accordance with the invention.

FIG. 2A is a side elevation of an alternative embodiment of a light emitting treatment system in accordance with the invention.

FIG. 2B is a side elevation of an alternative embodiment of a light emitting treatment system in accordance with the invention.

FIG. 3 is a diagrammatic side elevational representation of a germicidal lamp taken in a plane perpendicular to the longitudinal axis of the germicidal lamp to illustrate radiation emitted from the germicidal lamp when in that plane.

FIG. 4 is a top view of a food processing station comprising a conveyor and including light emitting treatment systems.

FIG. 5 is a side view, in cross-section, of a food processing station for coating food products with granular material and including light emitting treatment systems.

FIG. 6 is a top view of a food processing station comprising a packager and including light emitting treatment systems.

FIG. 7 is a diagram of a food processing station comprising an extruder and including light emitting treatment systems.

### DETAILED DESCRIPTION OF THE INVENTION

Throughout this description, the embodiments and examples shown should be considered as exemplars, rather than limitations on the apparatus and methods of the present invention.

When food items are produced by machinery, byproducts, residue and food product pieces may accumulate on the food processing equipment. In addition, food processing workers may release germs and other microorganisms and contaminants in the air surrounding the food processing machinery, and raw food ingredients may deposit bacteria, germs and other microorganisms and contaminants onto the food processing machinery. Germicidal lamps that emit ultraviolet (UV) radiation may be used to treat the food processing facility by both treating the food processing machinery and removing some of the potentially harmful germs, bacteria, other contaminants and the like from the food processing machinery and the air surrounding the food processing machinery.

As used herein, the terms food processing system, station, machinery, device, and other similar terminology refer to any automated or partially automated food handling, preparing, packaging, cooking, mixing, baking, and similar systems known to those skilled in the art. The food processing systems may package or otherwise place the food in a container to be delivered to a point of further distribution and eventual sale. The packaging described herein includes, but is not limited to, plastic bottles, cans made from aluminum and other metals, polystyrene trays, paper trays, paper bags, plastic bags, cardboard boxes, and the like known to those skilled in the art.

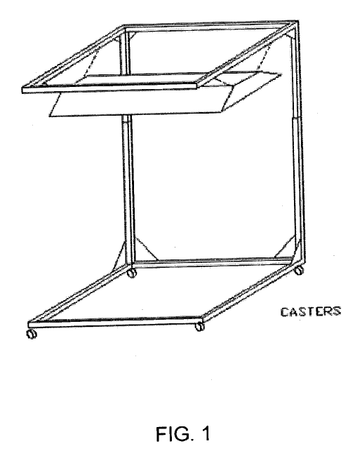

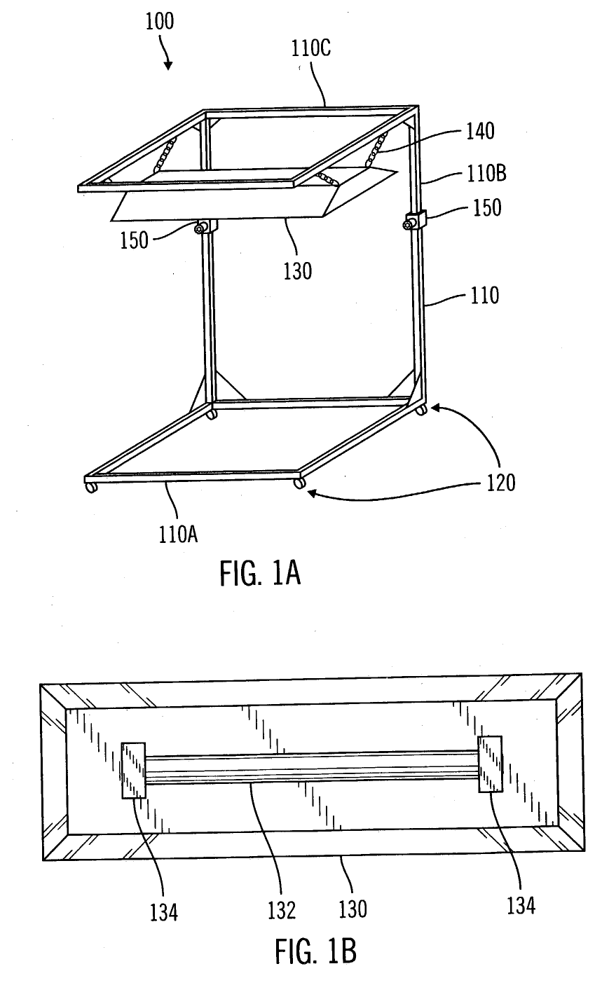

Referring now to FIG. 1A, a side elevation of a light emitting treatment system is shown. In this embodiment, light emitting treatment system 100 includes a frame 110 which has a bottom portion 110A and a vertical portion 110B which supports a top portion 110C. Frame 110 may be made of any sturdy and durable material such as metals, plastics, and the like. Bottom portion 110A and top portion 110C may be generally parallel to one another, and vertical portion 110B may be perpendicular to both top portion 110C and bottom portion 110A. Vertical portion 110B may include two or more interconnected tubes or the like which allow for the overall height of the light emitting treatment system to be adjusted.

In one embodiment, the vertical portion 110B may include two or more interconnected tubes that fit inside one another such that the diameter of each of the ends of the interconnected tubes may fit inside its neighboring tubes. When two or more interconnected tubes are used in vertical portion 110B, a locking bolt, clamp, or other securing device 150 may be included near the intersection of each of the interconnecting tubes. The securing device may be made out of a strong durable material such as metals, plastics, resins, combinations of these, and the like. The securing device may be loosened and tightened, unlocked and locked, etc. to allow for the sliding of the interconnected tubes in a vertical manner such that the height of the light emitting treatment system may be adjusted for a particular application, configuration or environment.

The light emitting treatment system may be made mobile by the use of casters 120 coupled to bottom portion 110A. Other low friction floor interfaces and techniques may be used to make the light emitting treatment system movable, such as, for example, wheels, bearings, sliders and the like. The casters, sliders, wheels or other low friction floor interfaces may be constructed out of durable materials including metals, plastics, resins, rubbers, combinations of two or more of these, and the like. The casters and other moving means may include a locking device (not shown) by which the light emitting treatment system may be held in a stationary position.

In the depicted embodiment, reflector housing 130 is coupled to the upper portion 110C by chains 140. Chains 140 may be used to adjust the hanging distance of the reflector housing 130 from upper portion 110C. In addition, the chains 140 may be adjusted to control the angle of the UV radiation directed from the lamp of the light emitting treatment system by moving the reflector housing 130 with relation to upper portion 110C. In this way, the light emitting treatment system may be adjusted to provide maximal UV radiation emission to a desired food processing station or device. In similar embodiments, other means for direction control may be used. For example, metal or other durable, heat resistant, cold resistant cable, line or wire may be used to couple reflector housing 130 to the upper portion 110C.

FIG. 1B is a view of an embodiment of the reflector housing 130 of a light emitting treatment system in accordance with the invention. Reflector housing 130 includes a lamp 132 coupled to sockets 134. Sockets 134 both hold lamp 132 in place and distribute power to the lamp. To achieve this, the sockets 134 are coupled to the reflector housing 130 and to a power source (not shown). In one embodiment, the lamp 132 is a UV emitting lamp that emits UVC radiation. In one embodiment, lamp 132 emits 40 watts of C-band ultraviolet irradiation so that the ultraviolet irradiation on the contaminated surface has an energy of at least 30 μWs/cm². Lamps of 110 watts have also been used. In these embodiments, ultraviolet radiation is produced substantially at 253.7 nm.

In various embodiments, lamp 132 may be one-sided or two-sided and may be coupled to the reflector housing 130 by one or two sockets 134. In one embodiment the light emitting treatment system includes a single lamp, although, in various other embodiments, the light emitting treatment system may include two, three, four or more lamps. The number of lamps used may be dependent on the size of the light emitting treatment system and/or the size of the area which is to be treated by the system. The reflector housing may be made of or coated with a reflective metal such as aluminum or other suitably reflective metal or other material. Coincidently, aluminum has in excess of 60% reflectivity for the primary UV emission line which has a wavelength of 253.7 nm. However, the method of the invention is also applicable to other materials which are relatively good reflectors of UV's primary emission line.

The lamp 132 and sockets 134 serve the same function as and contain similar components as the tube, tube base and other related components described in the following: U.S. Pat. No. 5,334,347 entitled “Electric Discharge Device”; U.S. Pat. No. 5,817,276 entitled “Method of UV Distribution In An Air Handling System”; U.S. Pat. No. 5,866,076 entitled “Single-Ended Germicidal Lamp for HVAC Systems”; and U.S. Pat. No. 6,280,686 entitled “Control of Health Hazards in an Air Handler,” which are co-owned with this application.

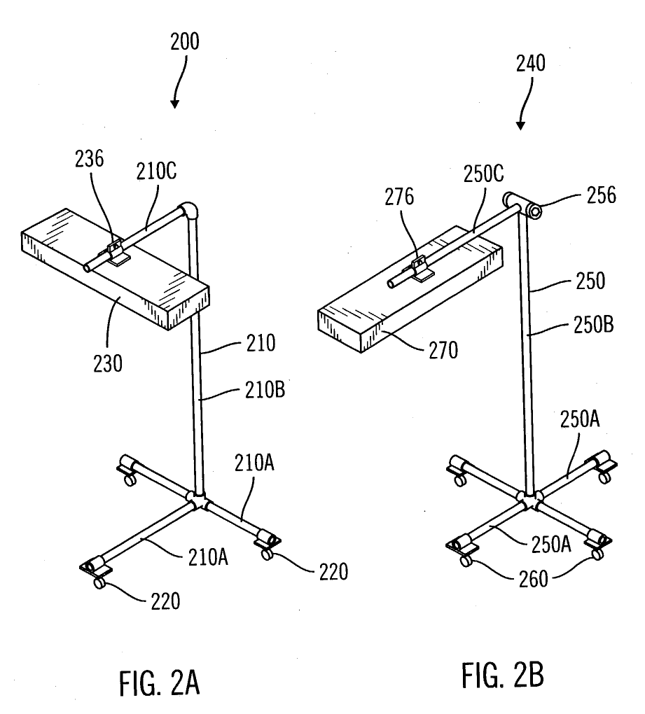

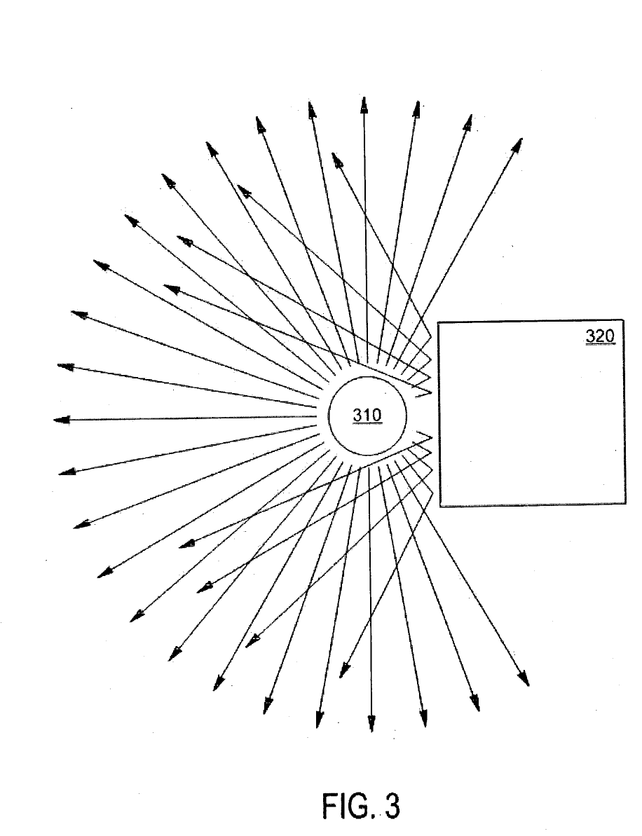

FIG. 2A is a side elevation of an alternative embodiment of a light emitting treatment system in accordance with the invention. In this embodiment, light emitting treatment system 200 may include frame 210 which has three bottom legs 210A and a vertical portion 210B which supports top portion 210C. Bottom portion 210A and top portion 210C are generally parallel to one another, allowing stability of the frame 210 while reducing the likelihood that the frame 210 will collide with support legs in the food plant. The vertical portion 210B is perpendicular to both top portion 210C and bottom portion 210A. In this embodiment, top portion 210C is fixedly coupled to vertical portion 210B.

The light emitting treatment system may be made mobile by the use of casters 220 coupled to bottom portion 210A. As discussed above regarding FIG. 1A, other techniques may be used to make the light emitting treatment system movable. The casters may include a locking device (not shown) by which the light emitting treatment system may be held in a stationary position.

In the depicted embodiment, reflector housing 230 is generally rectangular and is coupled in a fixable position to the upper portion 210C by clamp 236 or other securing means. In one embodiment, the clamp 236 may be adjustable so that the reflector housing 230 may rotate about upper portion 210C. In this way, UV radiation may be directed to a food processing device that is above, below or to the side of the lamp of the light emitting treatment system. As shown, the light emitting treatment system will disperse UV radiation to a food processing device below the lamp included in the light emitting treatment system. When the reflector housing is positioned such that the lamp is facing upwards, the light emitting system will disperse UV radiation to a food processing device located above the lamp. Similarly, when the reflector housing is positioned such that the lamp is facing sideways, the light emitting treatment system will disperse UV radiation to a food processing device located next to or beside the lamp.

FIG. 2B is a side elevation of an embodiment of a light emitting treatment system in accordance with the invention. In this embodiment, light emitting treatment system 240 includes a frame 250 having four bottom legs 250A and a vertical portion 250B which supports top portion 250C. The light emitting treatment system may be made mobile by the use of casters 220 coupled to bottom legs 250A. As discussed above regarding FIG. 1A, other low friction floor interfaces and techniques may be used to make the light emitting treatment system movable. The casters may include a locking device (not shown) by which the light emitting treatment system may be held in a stationary position.

In the depicted embodiment, reflector housing 270 is generally rectangular and is movably coupled to the upper portion 250C by locking clamp 276. In one embodiment, the locking clamp 276 may be adjustable so that the reflector housing 270 may rotate about upper portion 250C. In this way, the locking clamp allows a user to control the direction of UV radiation emitted to a food processing device that is above, below or to the side of the lamp included in the light emitting treatment system.

In this embodiment, the light emitting treatment system is further adjustable by using bracket 256 which connects vertical portion 250B and top portion 250C. Bracket 256 may be a hinge or other adjustable securing device. The upper portion 250C and the reflector housing 270 may be moved to increase or decrease the angle between upper portion 250C and vertical portion 250B. In this way, UV radiation dispersion and direction may be further controlled so that it is optimally aligned with and directed at food processing devices. In a similar embodiment, vertical portion 250B, bracket 256 and top portion 250C may be replaced by a single goose neck portion (not shown).

In other embodiments, the bottom portion 250B of frame 250 of the light emitting treatment system may have a generally solid bottom of any shape such as, for example, but not limited to, round or square. In these embodiments, the weight of bottom portion 250A should be sufficient to counter-balance the reflector housing 270 and upper portion 250C. In such an embodiment, bottom portion 250A may include or be augmented by metal, sand, or other material which will stabilize the light emitting treatment system when the reflector housing containing the lamp is positioned outward from vertical portion 250B, as shown in FIG. 2B. In a related embodiment, so that the light emitting treatment system may be mobile while also providing a stable base for extension of and positioning of the reflector housing, a tank which may be filled with water, sand or other substance may be added to bottom portion 250A. In this way, when the light emitting treatment system may be made heavier and thus more stable by adding water or sand, and be made lighter and thus more mobile by removing water or sand from the tank. Further, retractable wheels, sliders, casters or the like may be included in the base to allow for ease of mobility.

Although not shown in FIGS. 1A, 1B, 2A and 2B, the lamp in the reflector housing receives electrical power from a power cord that is coupled to the socket or sockets in the reflector housing. In various embodiments, the power cord may be internal to the support frame or may be wrapped around or otherwise coupled externally to the support frame. In one embodiment, the power cord may be plugged into a local electrical outlet. In another embodiment, the power cord may be connected to a battery or battery pack which may be set adjacent to or on the bottom portion of the frame of the light emitting treatment system, may be attached to the food processing station, or may be placed at another location within the food processing facility.

The light emitting treatment systems described herein may be used to treat food processing stations. A food processing station in a food processing plant is selected for treatment. The food processing station may have a contaminated surface upon which are disposed plural microorganisms and/or a dirty surface that requires cleaning. A light emitting treatment system comprising a germicidal lamp to emit ultraviolet light, a support structure for the germicidal lamp and a direction control device is provided adjacent to the food processing station. The germicidal lamp typically has a direction of ultraviolet irradiation output controllable by the direction control device, and the direction control device may be set in one of many fixable positions. The germicidal lamp may be moved proximate to the food processing station and near to the contaminated surface. The direction control device of the light emitting treatment system apparatus may be set to one of the fixable positions to direct ultraviolet irradiation from the germicidal lamp onto the contaminated surface of the food processing station. The light emitting treatment system may be connected to a power source such that when the system is energized, the lamp emits ultraviolet light to irradiate the contaminated surface of the food processing station with the ultraviolet light to render a minimum percentage of the microorganisms harmless.

FIG. 3 is a diagrammatic side elevational representation of a germicidal lamp taken in a plane perpendicular to the longitudinal axis of the lamp to illustrate radiation emitted from the germicidal lamp when in that plane. In determining the spatial relationship between the germicidal lamp and the target surface, the objective is to obtain a uniform distribution of UV radiation across the surface. It has been determined that, for a lamp which is positioned in accordance with the invention, the spatial distribution of UV radiation follows precisely that of a diffuse area source. It can be seen that although the germicidal lamp 310 is a source of radiation, the base 320 is effectively a secondary (reflected) source of UV radiation. The diffuse radiation of the germicidal lamp 310 and diffuse reflection is therefore defined as a near field effect, not as an inverse square law. Put another way, when the lamps 310 are positioned in sufficient proximity to the target surface, the intensity of UV radiation from the lamps 310 striking the target surface is, to a degree, independent of the distance of the lamps 310 from the target surface.

As shown in FIG. 3 the photons emitted from a particular point on the germicidal lamp 310 radiate in all directions. Because FIG. 3 is an elevational view, the global radiation of these photons is not shown. These photons would, however, also radiate outwardly and inwardly from the plane of the paper upon which the planar representation is illustrated and from all surfaces of the lamp 310. In addition, to increase the photons applied to the target surface, a germicidal lamp with a reflector is utilized. The reflector may be incorporated in reflector housing 320.

The adjustability of the lamps described herein, and in particular with regard to FIGS. 1A, 1B, 2A and 2B, allows for obtaining optimum positioning of the germicidal lamps with respect to the food processing devices. That is, as described herein, the height of the light emitting treatment system may be adjusted so that the lamp may be positioned a desirable distance from the food processing station. Similarly, the angle of the lamp may be adjusted in relation to the food processing system surface by positioning the light emitting treatment system described herein. This adjustability allows for controlling the direction of the UVC radiation emitted from the lamp of the light emitting treatment system.

Although there has been relatively little research on fly-by kill rates, it is known that some organisms require a higher dosage than others. To achieve a higher dosage, either UV power and exposure time may be increased. Exposure time may be increased, for example, by using more lamps or by slowing down the food processing equipment (e.g., conveyor belts).

In one embodiment, to provide a uniform distribution of photon energy through the deepest part of a food processing station, depending on its height and width, several lamps may be selected and positioned in the light emitting treatment system at “lamp to lamp” distances and “lamp to target surface” distances so that the minimum photon energy striking the leading edge of the target surfaces is preferably 716 μW/cm2 at the closest point and through placement, not less than 60% of that value at the farthest point. When positioned in this manner, nearly equal amounts of energy will also strike other areas of the food processing station, either directly or indirectly. The particular position of a light emitting treatment system relative to a target surface may also depend on the capabilities and characteristics of the germicidal lamp used in the light emitting treatment system.

The light emitting treatment system described herein may be used to kill molds, bacteria, and other unwanted organisms which may be present on food processing equipment. Our research has shown that the ionizing radiation from the germicidal lamps in the light emitting treatment system can be a key element in the killing and degradation process of microorganisms found in food processing stations. An ion is a particle formed when a neutral atom or group of atoms gains or loses one or more electrons. An atom that loses an electron forms a positively charged ion, called a cation and an atom that gains an electron forms a negatively charged ion, called an anion. Our scientific testing has established that the dead microorganisms then further undergo damage through this free radical process. Absorption of UVC energy leads to the formation of radical cations, anions and electrons, and electronically excited molecules. One reason is that about 70% of the energy is absorbed by the available moisture and about 30% by organic matter and other solutes. Water absorption of UVC leads to the formation of oxygen/hydrogen radicals or hydroxyls, solvated electrons and hydrogen atoms which are all very safe to humans and the environment. This process is similar to that produced by outdoor sunshine. In these processes, the atoms are separated, thus disassociating individual whole molecules to produce individual radicals to the original structure. These water-derived radicals are all highly reactive and atomically degrade (vaporize) organic material.

Only after continued study did we learn that the degradation process continues on the dead microorganisms as well as any residual organic nutrients. In time, exposed surfaces become organically cleaner. We have observed this effect on severely encrusted surfaces in as little as four weeks of continuous operation.

When a light emitting treatment system is utilized as described herein, total flux density between exposed parallel surfaces is at its highest. As such, microorganisms that are not defused on the surfaces and killed are mostly killed in the air due to the increased flux density from the resulting irradiation and lack of shadows. This reduces (kills) airborne microorganisms by as much as 90% on a single pass, reducing the incidence of airborne transmitted infections including such diseases as measles, chicken pox, whooping cough, common colds, influenza and tuberculosis which may have been introduced food workers, food handlers, food processing facility workers, etc.

Our research shows that UVC energy at 253.7 nm ionizes the organic bonds (as described above) of the typical materials deposited on food processing stations. UVC energy vaporizes these materials at the solid, molecular and atomic level.

The process of cleaning a target surface somewhat differs from the process of controlling the presence of surface and airborne microorganisms. The goal in cleaning the target surface is to eliminate a certain amount of organic matter. In contrast, the goal in controlling the presence of surface and airborne microorganisms is to sufficiently kill those microorganisms which are likely to affect health. In some applications, placement of the light emitting treatment system may vary depending on the goals.

The light emitting treatment system may be placed so that the lamps which emit UVC may be positioned from the target surface of the food processing station at a distance to maximize the effects of the UVC radiation. This distance is determined by determining the length of the centerline of the light string of the lamp or lamps in the light emitting treatment system. In one embodiment, it is preferable that the light emitting treatment system be placed a distance corresponding to 80% of the distance of the light string centerline of the lamps in the light emitting treatment system. For example, if the centerlines were 24″, then the distance from the food processing station should be approximately 20″. In addition, the light emitting treatment systems described herein may be placed distances corresponding to from 40% to greater than 100% of the length of the centerline of the light string.

The reflector in the light emitting treatment system serves to concentrate the energy produced and is aimed toward the target surface of the food processing station. In one embodiment, it has been found that positioning the light emitting treatment system approximately twenty inches from the target surface, in conjunction with appropriate germicidal lamp to lamp spacing in the light emitting treatment system, is particularly effective in inhibiting the growth of microorganisms on the target surfaces. In addition, the treatment of food processing stations using the light emitting systems described herein are also effective at closer and further distances, depending on the configuration of the food processing station, the configuration of the light emitting treatment systems, and the desired treatment.

Once positioned, the light emitting treatment systems may be run until the target surface is sufficiently treated. Once the target surface is sufficiently treated, the light emitting treatment system may be run continuously or intermittently, as required to maintain the cleanliness of and control the presence of germs and other microorganisms on the target surface.

When a food processing station is new, it is desirable to maintain it in the “as new” condition. Using the light emitting treatment systems as described herein may keep the target surface sufficiently clean indefinitely. This may result in keeping the food processing station up and running, thus reducing the down time needed to separately clean the food processing station.

Once the germicidal lamps are installed and turned on:

Contaminants are ionized and degraded (vaporized).

The light emitting treatment systems keep the target surfaces in this condition for the life of the food processing station.

The process is not destructive to the target surface or any other inorganic material.

The process requires no hazardous chemicals.

The process is environmentally friendly, as it adds nothing to the air or drainage system.

The light emitting treatment systems may do the job continuously without shutting down the food processing system or vacating the food processing plant.

An installation of the light emitting treatment systems can cost less than sporadic cleaning of the food processing system.

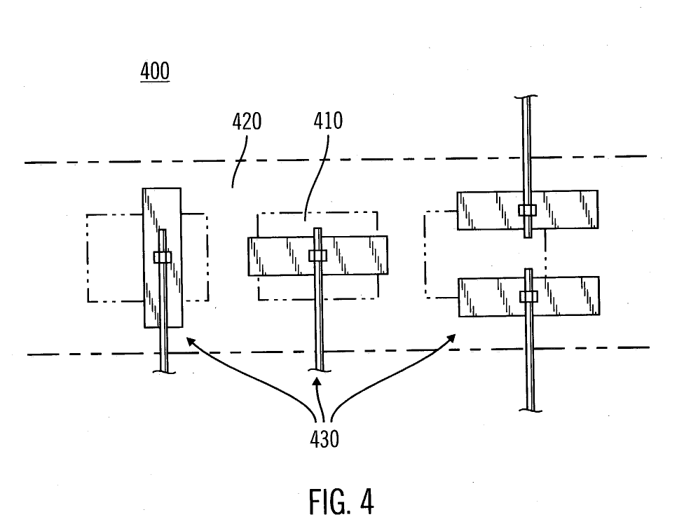

FIGS. 4-7 show several types of food processing stations and the use of light emitting treatment systems for treating the food processing stations by cleaning the food processing stations and by killing surface and airborne germs, bacteria, and the like. FIG. 4 is a top view of a food processing station 400 comprising a conveyor 420 and including light emitting treatment systems 430. Objects 410 are disposed on conveyor 420, and may be food products or packaging containers. As shown, both the food products, the packaging containers and the conveyor are all recipients of the output of the light emitting treatment systems. In this way, the food products, the packaging containers and the conveyor are all cleaned and made sufficiently germ-free by the lamps. In another embodiment, in those situations where the food product or the packaging containers are delicate and/or degrade when subjected to the UV radiation output of the lamps, one or more lamps may be placed toward the beginning of the conveyor before the food products or the packaging containers have been placed on the conveyor, and may be placed toward the end of the conveyor after the food products or the packaging containers have been removed from the conveyor. In addition, the lamps may be placed under the conveyor facing up such that the conveyor is bathed in UV radiation from the lamps on a return trip.

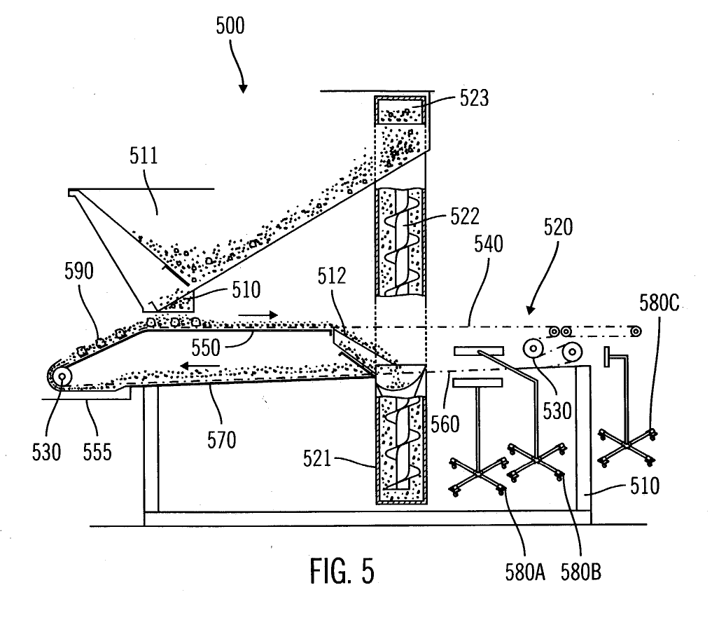

FIG. 5 is a side view, in cross-section, of a food processing station for coating food products with granular material and including light emitting treatment systems. Food processing station 500 may be used for coating food products with breadcrumbs or other granular material. Food processing station 500 comprises an open conveyor belt 520, indicated by dot-dashed lines, which is guided around rollers 530. The conveyor belt 520 may be made from wire material and has a top part 540 beneath which a top guide plate 550 extends. Other kinds of materials known to those skilled in the art may be used for the conveyor belt. The conveyor belt 520 has a bottom part 560 beneath which a bottom guide plate 570 extends.

The direction of rotation of the conveyor belt 520 is to the right in FIG. 5, as indicated by arrows. At the left-hand end of the conveyor belt 520, the bottom guide plate 570 merges into a diverter plate 555 which lies at a lower level than the bottom guide plate 570 and runs in a curve around the corresponding roller 530. The granular material which is supported on the bottom guide plate 570 is carried along by the conveyor belt 520 which is inherently permeable to the material, and via the diverter plate 555 is fed to the right over the top guide plate 550. On the left-hand section of the top guide plate 550, the food products 590 are moved onto the layer of coating material which has already been formed. The food products 590 emanate from a further conveyor belt which is not shown.

The products 590 then pass beneath the outlet 510 of the storage hopper 511, where a further quantity of granular material is positioned on the top side of the food products 590. As the top part 540 of the conveyor belt 520 moves further to the right, the products 590, which are now fully coated, are removed and the granular material falls through the conveyor belt 520 onto the screen plate, which is denoted overall by 512.

The relatively coarse material passes into a sleeve 521 in which there is a screw conveyor 522. By means of this screw conveyor 522, the material is pumped upwards to an opening 523 in the top section of the storage hopper 510, in such a manner that the said material can then be applied once again to the top side of the products 590.

To treat conveyor belt 520, a light emitting treatment system may be placed under the belt shown by lamp 580A so that the outer surface of the belt is treated on a return trip. Similarly, a lamp may be placed as shown by lamp 580B so that the inner surface of conveyor belt 520 is treated on a return trip. In addition lamp 580C may be placed adjacent to and to the side of where belt 520 passes along rollers 530 at the point where the belt begins its return trip. In addition, light emitting treatment systems may be placed and aligned with other surfaces within food processing station 500, such as the diverter plates, storage hopper 511, screw conveyor 522, etc.

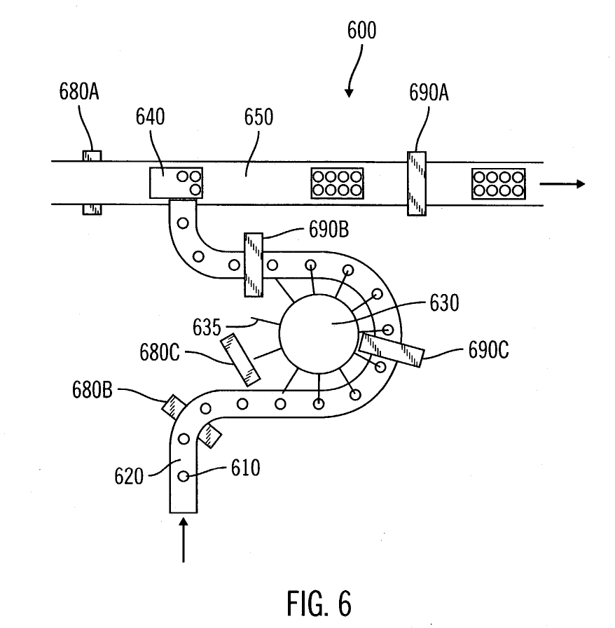

FIG. 6 is a top view of a food processing station 600 comprising a packager and including light emitting treatment systems. As shown, empty containers 610 are transported via a conveyor 620 to be filled as they pass through a filling station 630, which comprises a plurality of filling tubes 635. Once filled, individual containers 610 are packaged into multi-pack cartons 640. Multi-pack cartons 640 are then transported via conveyor 650 to be palletized.

To treat conveyor 650, a light emitting treatment system may be placed under the belt shown by lamp 680A so that the outer surface of the belt is treated on a return trip. Similarly, to treat conveyor 620, a light emitting treatment system may be placed under the belt shown by lamp 680B so that the outer surface of the belt is treated on a return trip. In another embodiment, a lamp may be placed at the beginning or end of the length of the conveyor belt so that the cartons are not subjected to UV radiation from the lamp. A lamp may be placed as shown by lamp 680C to treat the external surfaces of filling tubes 635.

In yet another embodiment, lamps 690A, 690B and 690C may be placed over conveyors 620 and 650. In one version of this embodiment, a sensor may be used to coordinate the lamp with the conveyor belts in such a way so UV radiation is only emitted onto vacant portions of the conveyor belt, that is, when a food item or container is not on the particular portion of the conveyor that is within the throw area of the UV radiation from the lamp.

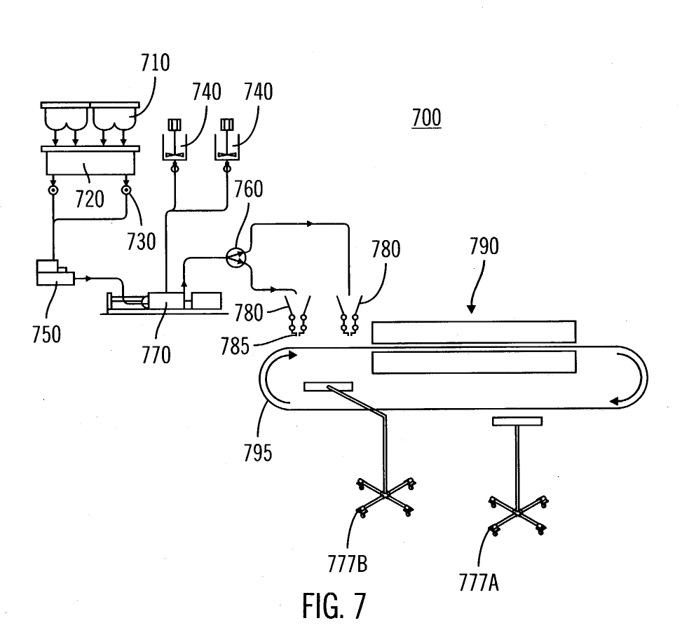

FIG. 7 is a diagram of a food processing station comprising an extruder and including light emitting treatment systems. A food product is extruded from food processing station 700. The various basic ingredients, such as meat, cereals, water and fat, are mixed in the mixer 710. The mixer 710 feeds into a storage tank 720. Pumps 730 allow the mixture obtained to be taken continuously to a stuffer 750. The food processing station 700 further comprises an emulsifying device 770, coloring-solution storage tanks 740, a three-way valve 760, mechanical extruders 780 and a steam oven 790.

In operation, stuffer 750 feeds the basic ingredients to the emulsifying device 770. Coloring solutions from tanks 740 enter the emulsifying device 770. The valve 760 is left open for a period and the paste feeds the mechanical extruder 780. The extruder 780 may have a die 785 through which the paste passes in order to form pieces which drop onto a conveyor belt 795 of the steam oven 790. The oven 790 cooks the pieces.

To treat conveyor 795, a light emitting treatment system may be placed under the belt shown by lamp 777A so that the outer surface of the belt is treated on a return trip. In addition, the lamp of a light emitting treatment system may be placed over the inside of the belt shown by lamp 777B so that the inner surface of the belt is treated on a return trip. It has been found that the lamps work well in environments ranging from −30° to 150° F. In addition, light emitting treatment systems may be placed and aligned with other surfaces within food processing station 700, such as adjacent to pumps 730, stuffer 750, valve 760, etc.

In one embodiment, the light emitting treatment system may be augmented by or include one or more mirrors to redirect or channel the UV radiation to a desired location that may not be reached directly by the light emitting treatment system. In another embodiment, the light emitting treatment system may include a light pipe to disperse UV radiation into narrow or tight fitting locations or to direct a small diameter of light onto a desired object or machinery. In food processing stations, it may be desirable to direct a stream of UV radiation onto a nozzle or other small area. In addition, the light pipe embodiment of the light emitting treatment system may be used to snake through densely packed food processing machinery. In light pipes, UV rays enter a tube, being either solid or hollow, and reflect from the walls an indeterminate number of times until they emerge from the tube. The light pipe may be made from and include optical glass or poly methyl methacrylate, also known as PMM and Plexiglass®, as is well known to those skilled in the art.

Although the light emitting treatment system has thus far been discussed regarding food processing, the light emitting treatment system may also be used to eradicate mold and household germs from bathrooms, kitchens and other areas in homes. In one embodiment, the light emitting treatment system may be aligned so that UVC radiation is dispersed toward bathroom fixtures, drains, sinks, showers, as well as kitchen sinks, and other similar fixtures in these and other rooms of a house.

In addition, the light pipe embodiment may be used to eradicate mold and microorganisms from inside walls within a home or commercial building. In this embodiment, access may be obtained to the internal portion of a wall by, for example, cutting a hole in a wall large enough for entrance of a light pipe. The light emitting treatment system having a light pipe may be inserted into the wall to treat the surface and air within the wall. In way, a single hole or access point may allow for treating an area within a wall. The light emitting treatment system may also be used for treating the underside of floors of houses, in basements, in crawls spaces and the like in any of the embodiments described herein.

Although exemplary embodiments of the present invention have been shown and described, it will be apparent to those having ordinary skill in the art that a number of changes, modifications, or alterations to the invention as described herein may be made, none of which depart from the spirit of the present invention. All such changes, modifications and alterations should therefore be seen as within the scope of the present invention.

It is claimed:

1. A method of treating a food processing plant comprising
selecting a food processing station in the food processing plant for treatment, the food processing station having a contaminated surface
providing a light emitting treatment system comprising a germicidal lamp to emit ultraviolet light, a support structure for the germicidal lamp and a direction control device, wherein the germicidal lamp has a direction of ultraviolet irradiation output controllable by the direction control device, the direction control device having a plurality of fixable positions
moving the germicidal lamp apparatus proximate to the food processing station and near to the contaminated surface
setting the direction control device of the light emitting treatment system apparatus to one of the fixable positions such that ultraviolet irradiation from the germicidal lamp will be directed onto the contaminated surface of the food processing station
connecting the germicidal lamp to a power source and energizing the germicidal lamp to emit ultraviolet light
irradiating the contaminated surface of the food processing station with the ultraviolet light to treat the contaminated surface.

2. The method of claim 1 wherein ambient air temperature is between 10° and 110° F.

3. The method of claim 1 wherein the contaminated surface of the food processing station has a temperature of between 10° and 110° F.

4. The method of claim 1
wherein the contaminated surface has disposed thereon plural microorganisms
wherein the irradiating renders a minimum percentage of the microorganisms harmless.

5. The method of claim 1 wherein the germicidal lamp emits at least 40 W of C-band ultraviolet irradiation.

6. The method of claim 1 wherein the ultraviolet radiation on the contaminated surface has an energy of at least 30 μWs/cm².

7. The method of claim 1 wherein the light emitting treatment system is placed from two to six feet from the contaminated surface.

8. The method of treating a food processing plant of claim 1 wherein the light emitting treatment system further comprises a low friction floor interface.

9. The method of treating a food processing plant of claim 8 wherein the low friction floor interface comprises wheels.

10. The method of treating a food processing plant of claim 8 wherein the low friction floor interface comprises sliders.

11. The method of treating a food processing plant of claim 1, wherein the direction control device comprises a height adjuster.

12. The method of treating a food processing plant of claim 1, wherein the direction control device comprises an angle adjuster.

13. The method of treating a food processing plant of claim 1, wherein the minimum percentage is 99%.

14. The method of treating a food processing plant of claim 1, wherein the food processing station comprises a conveyor belt.

15. The method of treating a food processing plant of claim 1, wherein the food processing station comprises a bottle filler.

16. The method of treating a food processing plant of claim 1, the irradiating step comprising eviscerating at least some of the microorganisms.

17. The method of treating a food processing plant of claim 1, the irradiating step comprising sterilizing at least some of the microorganisms.

18. The method of treating a food processing plant of claim 1, the irradiating step further comprising maintaining power to the lamp for a minimum period of time.

19. The method of treating a food processing plant of claim 1 wherein the ultraviolet light is substantially exclusively UVC.

20. The method of treating a food processing plant of claim 1 wherein the light emitting treatment system further comprises a power supply disposed electrically between the lamp and the power source.

21. The method of treating a food processing plant of claim 1 wherein the light emitting treatment system further comprises a battery, and the battery comprises the power source.

22. The method of treating a food processing plant of claim 1 wherein the light emitting treatment system further comprises a power cord, and the connecting step comprises connecting the power cord to an external power source.

23. A method of treating a food processing station, comprising the steps of:
positioning a light emitting treatment system such that the output of a germicidal lamp included therein will fall on a desired surface of the food processing station
energizing the germicidal lamp to emit substantially uniformly distributed ultraviolet radiation across the desired surface of the food processing station.

24. The method of claim 23 wherein the positioning comprises
setting a direction control device of the light emitting treatment system to one of a plurality of fixable positions such that ultraviolet irradiation from the germicidal lamp will be directed onto the contaminated surface of the food processing station.

25. The method of claim 23 wherein the energizing comprises
irradiating the desired surface of the food processing station with the ultraviolet light such that a minimum percentage of the microorganisms are rendered harmless.

26. The method of claim 23 wherein the germicidal lamp emits at least 40 W of C-band ultraviolet irradiation and the ultraviolet irradiation on the contaminated surface has an energy of at least 30 μWs/cm².

27. The method of claim 23 wherein the germicidal lamp emits ultraviolet radiation substantially at 253.7 nm and generates an insignificant quantity or less of ozone.
